(19) United States
(12) Patent Application Publication
(10) Pub. No.: US 2024/0150175 A1
(43) Pub. Date: May 9, 2024

(54) STANNOUS PYROPHOSPHATE, AND METHODS OF PRODUCING THE SAME

(71) Applicant: HONEYWELL INTERNATIONAL INC., Charlotte, NC (US)

(72) Inventors: Juergen Rudolph, Peine (DE); Christian Werner, Hannover (DE)

(73) Assignee: HONEYWELL INTERNATIONAL INC., Charlotte, NC (US)

(21) Appl. No.: 18/485,472

(22) Filed: Oct. 12, 2023

Related U.S. Application Data
(60) Provisional application No. 63/382,295, filed on Nov. 4, 2022.

(51) Int. Cl. C01B 25/42; A61K 8/24; A61Q 11/00

(52) U.S. Cl. CPC C01B 25/42; A61K 8/24; A61Q 11/00; A61K 2800/412

(57) ABSTRACT

Dental health care compositions including tin pyrophosphate and methods of producing the same are provided. In an exemplary embodiment, a method of producing tin pyrophosphate includes combining tin tetrafluoroborate with a pyrophosphate salt composition to produce a precipitate, where the precipitate includes tin pyrophosphate and a tetrafluoroborate salt. The tetrafluoroborate salt is then removed from the precipitate.

### CROSS REFERENCE TO RELATED APPLICATION

This application claims priority to U.S. Provisional patent application Ser. No. 63/638,5392 filed on Nov. 4, 2022, which is incorporated herein by reference.

### TECHNICAL FIELD

The present disclosure generally relates stannous pyrophosphate and methods of making the same. More particularly, the present disclosure relates to producing stannous pyrophosphate by reacting stannous boron tetrafluoride with a pyrophosphate salt composition.

### BACKGROUND

Stannous ion sources improve many oral care products with favorable clinical benefits, such as a reduction of gingivitis and a reduction of tooth demineralization from erosion. Stannous fluoride is a well known example of such a stannous ion source, and has been used for many years. However, stannous fluoride is somewhat instable in aqueous solutions due at least in part to the reactivity of the stannous ion. Stannous salts hydrolyze at pH values above 4, and then precipitate from solution. The precipitated form of the stannous salt may reduce the therapeutic properties.

Soluble stannous ions may also unfavorably react with certain rheological modifiers, such as some types of celluloses and gums. Such compounds may be considered incompatible soluble stannous ions, and these compounds are often used in dental health products.

Stannous pyrophosphate is known as a dentifrice polishing agent, and may overcome of the limitations mentioned above. Stannous pyrophosphate, which has the formula Sn₂P₂O₇, includes the tetravalent pyrophosphate ion and a divalent stannous cation (i.e., Sn(II).) Stannous pyrophosphate is substantially insoluble in water, especially in acidic conditions. However, the use stannous pyrophosphate has been limited by its high cost. Furthermore, a small particle size is desired for incorporation of stannous pyrophosphate into dental health care products. Several methods of producing stannous pyrophosphate have been described, but methods or producing high purity stannous pyrophosphate with a small particle size is still desirable.

Accordingly, it is desirable to find new stannous pyrophosphate production techniques that produce high purity product. In addition, it is desirable to find production techniques that produce stannous pyrophosphate with a small particle size suitable for incorporation into a dental heath care product. Furthermore, other desirable features and characteristics of the present embodiment will become apparent from the subsequent detailed description and the appended claims, taken in conjunction with this background.

### BRIEF SUMMARY

Dental health care compositions including tin pyrophosphate and methods of producing the same are provided. In an exemplary embodiment, a method of producing tin pyrophosphate includes combining tin tetrafluoroborate with a pyrophosphate salt composition to produce a precipitate, where the precipitate includes tin pyrophosphate and a tetrafluoroborate salt. The tetrafluoroborate salt is then removed from the precipitate.

A dental health care composition is provided in another embodiment. The dental health care composition includes tin pyrophosphate at a concentration of from about 96 to about 99.999 weight percent, based on a total weight of the dental health care composition. The dental health care composition also includes sodium tetrafluoroborate at a concentration of from about 10 to about 1,000 parts per million by weight, based on the total weight of the dental health composition.

Another method of producing tin pyrophosphate is provided in yet another embodiment. A tin tetrafluoroborate solution is provided that includes tin tetrafluoroborate and a tin tetrafluoroborate solvent, where the tin tetrafluoroborate solvent includes water. A Pyrophosphate salt is provided that includes a pyrophosphate salt solvent and tetrasodium pyrophosphate, where the pyrophosphate salt solution includes water. A pyrophosphate salt solution is adjusted to about 60 to about 85 degrees Celsius. The tin tetrafluoroborate solution and the pyrophosphate salt solution are combined to produce a precipitate that includes tin pyrophosphate and sodium tetrafluoroborate. The precipitate is rinsed with a rinsate to reduce a sodium tetrafluoroborate concentration to a level such that the boron concentration is less than about 100 parts per million by weight, based on a total weight of the precipitate.

### BRIEF DESCRIPTION OF THE DRAWINGS

The present embodiments will hereinafter be described in conjunction with the following drawing figures, and wherein:

FIG. 1 illustrates a technique for forming a tin tetrafluoroborate solution;

FIG. 2 illustrates a technique for forming a pyrophosphate salt solution;

FIGS. 3-5 illustrate different embodiments of techniques for forming a reaction product that includes tin pyrophosphate;

FIG. 6 illustrates an embodiment of washing a precipitate; and

FIG. 7 illustrates an embodiment of drying the precipitate.

### DETAILED DESCRIPTION

The following detailed description is merely exemplary in nature and is not intended to limit the various embodiments or the application and uses of the embodiments described herein. Furthermore, there is no intention to be bound by any expressed or implied theory presented in the preceding technical field, background, brief summary, brief description of the drawings, or the following detailed description or drawings.

Stannous pyrophosphate has a very low solubility in water, and is a relatively stable salt. Stannous pyrophosphate is sometimes referred to as tin pyrophosphate, and has the chemical formula of Sn₂P₂O₇. The tin 2+ cation is preferred to the tin 4+ cation, and is most prevalent in the reactions described below. In general, tin tetrafluoroborate, with a chemical formula of Sn(BF₄)₂, is combined with a pyrophosphate salt compound to produce the tin pyrophosphate and a tetrafluoroborate salt byproduct, which has the chemical formula of NaBF₄ in an embodiment where the pyrophosphate salt is a sodium pyrophosphate salt. The reactants may be in solution when combined, where the reactants are more soluble in polar solvents. The tin pyrophosphate is produced as a precipitate when formed in solution, and the size of the precipitate particles can be controlled as described below.

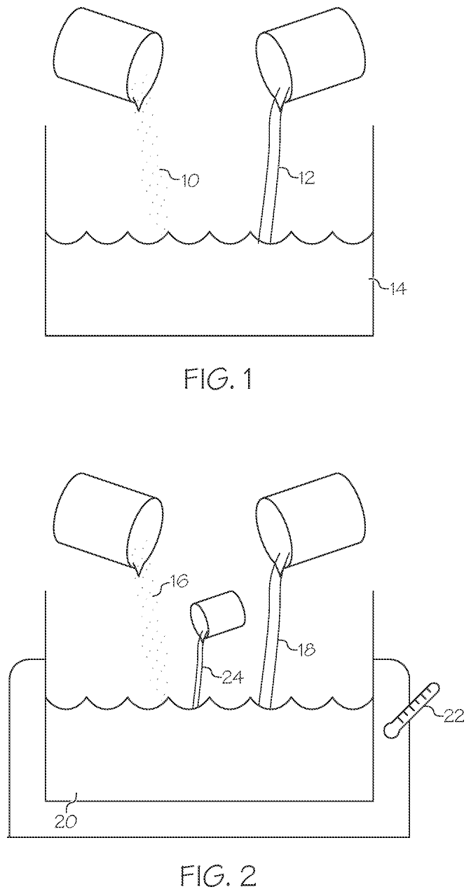

Reference is made to FIG. 1. Tin tetrafluoroborate 10 is available as a raw material, and can be purchased in sufficient quantities from various suppliers, often in a 50/50 weight percent aqueous solution, based on a total weight of the aqueous solution. Tin tetrafluoroborate 10 is typically used as a plating agent and/or a surface treating agent. Tin tetrafluoroborate 10 is a solid material with a melting point of greater than 130 degrees Celsius (° C.), and is miscible with water forming a colorless and stable aqueous solutions.

A tin tetrafluoroborate solvent 12 may be combined with the tin tetrafluoroborate 10 to form a tin tetrafluoroborate solution 14. The tin tetrafluoroborate solvent 12 includes water, and may include from about 50 to about 100 weight percent water, based on a total weight of the tin tetrafluoroborate solvent 12. In some embodiments, the tin tetrafluoroborate solvent 12 is from about 95 to about 100 weight percent water, but other components may be included in alternate embodiments. For example, alcohols or other polar compounds may be included in the tin tetrafluoroborate solvent 12. Exemplary alcohols have from 1 to 6 carbon atoms, but other alcohols or other types of solvent may also be used. In general, the tin tetrafluoroborate solvent 12 is capable of dissolving the tin tetrafluoroborate 10. In some embodiments, the tin tetrafluoroborate solution 14 is acidic, having a pH of less than 7, so the tin tetrafluoroborate solvent may also have an acidic pH. In some embodiments, the tin tetrafluoroborate 10 is formed by adding a tin salt to a tetrafluoroborate acid solution, and the tin tetrafluoroborate 10 is isolated by electrolysis. However, different manners of production may be utilized in alternate embodiments.

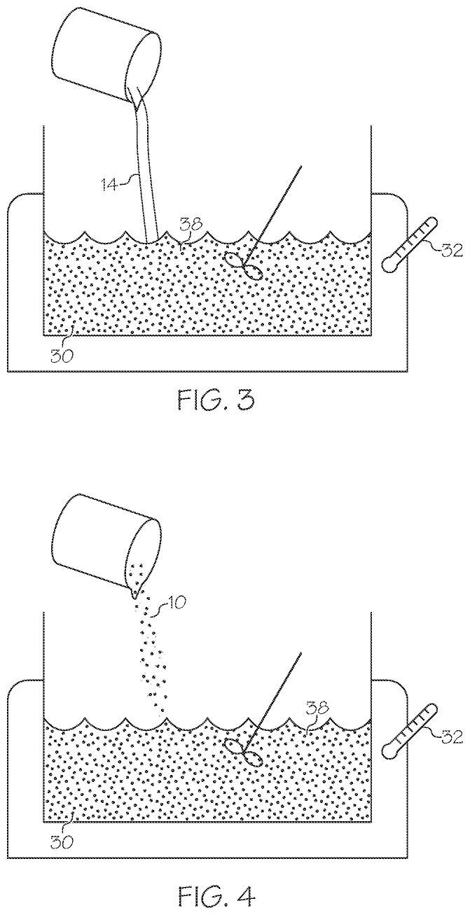

Referring to FIG. 2, the pyrophosphate salt compound 16 is combined with a pyrophosphate salt solvent 18 to form a pyrophosphate salt solution 20. The pyrophosphate salt compound 16 may comprise tetrasodium pyrophosphate, with the chemical formula of Na₄P₂O₇, but the sodium pyrophosphate compound 16 may also include disodium pyrophosphate, with a chemical formula of Na₂H₂P₂O₇. In other embodiments, the pyrophosphate salt may include metals other than, or in combination with, sodium as the cation of the salt. As non-limiting examples, the cation may include one or more of potassium, rubidium, calcium, magnesium, iron, or others. In an exemplary embodiment, the pyrophosphate salt compound 16 comprises tetrasodium pyrophosphate in an amount of from about 50 to 100 weight percent, based on a total weight of the pyrophosphate salt compound 16. Pyrophosphate salt compounds 16 are readily available commercially, in various grades and purities.

The pyrophosphate salt compound 16 is somewhat soluble in water, so the pyrophosphate salt solvent 18 may include water in an amount of from about 50 to 100 weight percent, based on the total weight of the pyrophosphate salt solvent 18. However, other solvents may also be included in the pyrophosphate salt solvent 18, such as alcohols or other solvents. Due to the limited solubility of the pyrophosphate salt compound 16 in the pyrophosphate salt solvent 18, the pyrophosphate salt solution 20 may be heated to facilitate dissolution. In an exemplary embodiment, the pyrophosphate salt solution 20 is heated to a pyrophosphate salt solution temperature 22 of from about 60 to abut 85° C., but other temperature ranges are also possible. Tetrasodium pyrophosphate has a solubility in water of 6.7 grams per milliliter (g/ml) at 25° C., but the solubility increases to 42.2 g/ml at 100° C. In an exemplary embodiment, the pyrophosphate salt compound 16 is present in the pyrophosphate salt solution 20 in an amount of from about 10 to about 500 grams per liter, or in an amount of from about 100 to about 300 grams per liter.

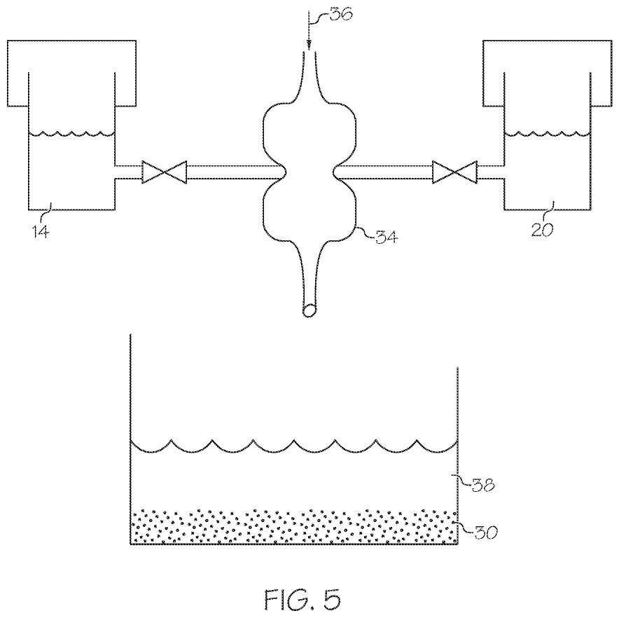

Referring to FIG. 3, with continuing reference to FIGS. 1 and 2, the tin tetrafluoroborate 10 is added to the pyrophosphate salt compound 16 to produce the tin pyrophosphate in a reaction product 38. In an exemplary embodiment illustrated in FIG. 3, the tin tetrafluoroborate solution 14 is added to the pyrophosphate salt solution 20, where the two compositions react and form the tin pyrophosphate that is included in a precipitate 30. The reaction of the tin tetrafluoroborate 10 and the pyrophosphate salt compound 16 may be controlled at a reaction temperature 32 of about 50 to about 100° C., but other reaction temperature ranges are also possible. For example, reaction temperature ranges of from about 50 to about 80° C., or from about 60 to about 70° C., may also be utilized. The tin tetrafluoroborate 10 may be added to the pyrophosphate salt solution 20 over an addition period of from about 5 minutes to about 12 hours, but other additions periods are also possible. For example, the addition period may be from about 20 minutes to about 2 hours, or from about 30 minutes to about 60 minutes in alternate embodiments. The reaction of the tin tetrafluoroborate 10 and the pyrophosphate salt compound 16 produces a tetrafluoroborate salt as a byproduct, such as sodium tetrafluoroborate when a sodium pyrophosphate salt is in the pyrophosphate salt compound 16. Therefore, the tetrafluoroborate salt is within the reaction product 38.

The tin tetrafluoroborate 10 and the pyrophosphate salt compound 16 react to produce tin pyrophosphate and a tetrafluoroborate salt product. A review of the chemical formulas allows a chemist to determine the stoichiometric quantity of the tin tetrafluoroborate 10 and the pyrophosphate salt compound 16, where the stoichiometric quantity is the theoretical amount where all of the reactants (tin tetrafluoroborate 10 and the pyrophosphate salt compound 16) are reacted in total to produce the products. In an exemplary embodiment, the tin tetrafluoroborate 10 and the pyrophosphate salt compound 16 are combined at about 100 percent of the stoichiometric amount, where 100% of the stoichiometric amount is the stoichiometric amount. The term “about 100 percent of the stoichiometric amount” means within about 5% of the stoichiometric amount in this description. In alternate embodiments, the tin tetrafluoroborate 10 is added in an amount of about 100 to about 125% of a stoichiometric amount for a reaction with the pyrophosphate salt composition 16, so the tin tetrafluoroborate may be added in excess of the stoichiometric amount. In yet another embodiment, the tin tetrafluoroborate is added in an amount of about 75 to about 100% of the stoichiometric amount. Other embodiments are also possible.

Many different embodiments for the combination of the tin tetrafluoroborate 10 and the pyrophosphate salt compound 16 are possible. For example, the tetrafluoroborate 10 may be added as a solid to the pyrophosphate salt solution 20, as illustrated in FIG. 4. In an alternate embodiment, the tin tetrafluoroborate solution 14 and the pyrophosphate salt solution 20 are simultaneously injected into a microjet reactor 34, as illustrated in FIG. 5. In an exemplary embodiment, a reaction gas 36 is added with the reactants, and a reaction product 38 is discharged, where the reaction product 38 includes the precipitate 30 with the tin pyrophosphate. Other alternate possibilities include the continuous simultaneous addition of the tin tetrafluoroborate 10 and the pyrophosphate salt compound 16 to a continuous reactor (not illustrated), where the tin tetrafluoroborate 10 and/or the pyrophosphate salt compound 16 may be added as a solid or as a solution. Other possible reaction mechanisms are also possible.

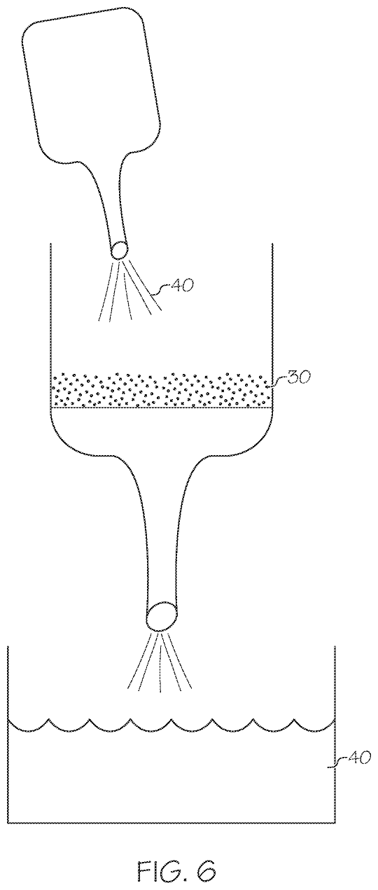

The precipitate 30 may be removed from the reaction product 38 by filtration, settling, centrifugation, or other separation techniques, as illustrated in an exemplary embodiment in FIG. 6 with continuing reference to FIGS. 1-5. The precipitate 30 may be washed to increase the purity of the tin pyrophosphate and to reduce the concentration of the tetrafluoroborate salt. In an exemplary embodiment, the precipitate 30 is washed by rinsing with a rinsate 40, but other techniques for removing the tetrafluoroborate salt are also possible. For example, the precipitate 30 may be re-slurried and re-filtered, the tin pyrophosphate could be further purified by recrystallization, or other purification techniques could be used for removing the tetrafluoroborate salt. In an exemplary embodiment wherein the precipitate 30 is rinsed, the rinsate 40 comprises water in an amount of from about 50 to about 100 weight percent, based on a total weight of the rinsate 40. The tin pyrophosphate is much less soluble in water than the tetrafluoroborate salt byproduct, so the tetrafluoroborate salt is preferentially dissolved and washed from the tin pyrophosphate in the precipitate 30 during the rinse or wash. The tin pyrophosphate has a specification of 100 ppm of boron in an exemplary embodiment, so the precipitate 30 may be washed until the tetrafluoroborate salt concentration is reduced to the point where the boron concentration is 100 ppm or less, based on a total weight of the precipitate 30 after drying. As such, the precipitate 30 may be washed until the concentration of the tetrafluoroborate salt is about 10 to about 1,000 ppm, measured based on the total weight of the precipitate 30 after drying. In an exemplary embodiment, the amount of rinsate 40 used is about 500 grams of water per 100 grams of tin pyrophosphate in the precipitate 30 to reduce the tetrafluoroborate salt to about 1,000 ppm or less, based on the total weight of the tin pyrophosphate in the precipitate 30. In alternate embodiments, about 300 grams, or about 400 grams of water are used per 100 grams of tin pyrophosphate. The rinse may be repeated until the impurities are reduced to a level sufficient for use in dental products. In an exemplary embodiment, the rinse may be repeated until the rinsate, after passing through the precipitate 30, has a conductivity of about 500 micro siemens or less. Other techniques may be utilized to verify the desired purity in alternate embodiments.

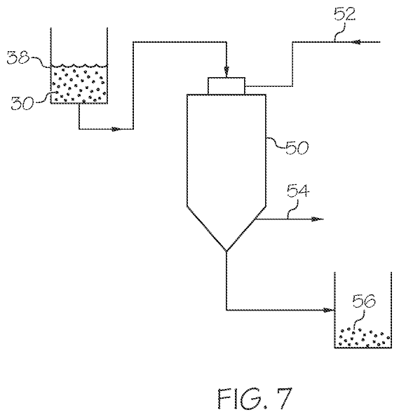

The precipitate 30 may be dried after being washed, such as in a spray drier, as illustrated in FIG. 7 with continuing reference to FIGS. 1-6. The precipitate 30 may be slurried or otherwise fed to a spray dryer 50. Drying gas 52, such as hot air, is fed into the spray dryer 50 with the precipitate 30, where the drying gas 52 exits the spray dryer 50 as an exhaust gas 54 and tin pyrophosphate 56 exits the spray dryer 50 as a solid product. The precipitate 30 may be dried using many other techniques, such as a fluidized bed, tray dryers, vacuum dryers, freeze dryers, and others. The type of dryer utilized may influence the particle size of the product, which includes the tin pyrophosphate 56.

The tin pyrophosphate 56 in the product may be present in a concentration of from about 96 to about 99.99 weight percent in the product, based on a total weight of the product, where the product may be a dental health care composition. The boron concentration may be from about 0.1 to about 100 ppm by weight, based on the weight of the product. The sodium tetrafluoroborate (or other tetrafluoroborate salts) may be present in the product at a concentration of from about 10 to about 1,000 ppm by weight, based on the total weight of the product, where the tetrafluoroborate salt typically remains in small quantities. In an exemplary embodiment, the presence of the sodium tetrafluoroborate (or other tetrafluoroborate salt) in the product is a strong indication that the tin pyrophosphate 56 was produced using tin tetrafluoroborate and a sodium pyrophosphate compound (or other pyrophosphate salt compound 16) as reactants. The tin pyrophosphate 56 product may include a wide variety of other impurities in various embodiments, and these impurities may result from the raw materials used. Exemplary impurities that may be present include trace elements such as arsenic (As), cadmium (Cd), cobalt (Co), mercury (Hg), nickel (Ni), lead (Pb), antimony (Sb), vanadium (V), chlorine (Cl), chromium (Cr), potassium (K), etc. In general, these trace elements may optionally be present at a concentration ranging from 0 to about 500 ppm.

It is desirable for the tin pyrophosphate 56 produced in the product to have a small average particle size to facilitate incorporation into oral health products as a dental health care composition. Various techniques can be incorporated into the production process to reduce the average particle size in the product. For example, the use of a spray dryer 50 can reduce particle size. Incorporate of an acid into the pyrophosphate salt solution 20 prior to the reaction can reduce the pH, which slows the reaction and can help reduce the average particle size. Phosphoric acid is an example of an acid that can be used, but other acids are also possible, such as hydrochloric acid, sulfuric acid, citric acid, acetic acid, etc. The acid may be incorporated into the pyrophosphate salt solution 20 in an amount of from about 0.01 to about 1.0 weight percent in an exemplary embodiment, and may lower the pH of the pyrophosphate salt solution 20 to a range of from about 10 to about 6, or from about 9.5 to about 7, or from about 9.5 to about 9 in various embodiments. In other embodiments, an antiscalant 24 can optionally be added to the pyrophosphate salt solution 20 in an amount of from about 0.05 to about 0.5 weight percent, or from 0.1 to about 0.5 weight percent, based on a total weight of the pyrophosphate salt solution 20. The antiscalant 24 may be selected from the group of citric acid, nitrilotris(methylene) triphosphonic acid (NTMP), etidronic acid (also known as hydroxyethylidene (1,1-diphosphonic acid)) (HEDP), phosphonobutane tricarboxylic acid, ethylenediamine tetra (methylene phosphonic acid), hexamethylene diamine tetra (methylene phosphonic acid), diethylenetriamine penta (methylene phosphonic acid) (DTPMP), and combinations thereof. The use of a micro-jet reactor 34 also can produce small particle sizes.

Testing has shown the tin pyrophosphate 56 in the product may have an average particle size D95 of from about 0.1 to about 100 microns, but higher particles sizes are also possible based on the materials used and the production process. The D95 particle size means that 95% of the particles are less than the stated particle size. In an exemplary embodiment, the particle size may be determined by laser diffraction through a suspension, but other techniques may be used in alternate embodiments. In an exemplary embodiment, the use of phosphoric acid, and/or other antiscalants 24 can reduce the particles size D95 of the tin pyrophosphate 56 in the reaction product 38 to about 0.1 to about 100 microns. The use of ultrasound on slurried precipitate 30 has also produced particles sizes D95 of from about 0.1 to about 100 microns. Furthermore, addition periods of about 5 to about 12 hours has produced particle sizes D95 of about 0.1 to about 100 microns.

Other techniques may also be utilized to reduce the particle size of the reaction product 38. For example, the precipitate 30 may be slurried and exposed to ultrasound procedures, high-speed mixing, homogenizing, and/or other rapid agitation techniques. Furthermore, the reaction product 38 can be milled, ground, or processed in other ways to reduce the particle size, or to sift the product and recover smaller particles. Increasing the addition period slows the reaction, and this can decrease particle size. The various approaches to reduce particle size have advantages and disadvantages to be considered and balanced. For example, milling or grinding use extra energy, and dusting issues may result. Increased dosing time slows the production process, which increases costs for manpower, depreciation, and other related costs. The cost of the antiscalants 24 must be considered, as well as the cost of maintaining an additional product in the production process.

### EXPERIMENTAL DATA

Several batches were made in the lab using the techniques described above, and the results are provided below. In Table 1, all charge quantities are in grams, unless otherwise stated.

TABLE 1
Part 1

| | Run 1 | Run 2 | Run 3 | Run 4 | Run 5 | Run 6 | Run 7 | Run 8 |
|---|---|---|---|---|---|---|---|---|
| Sn(BF₄)₂ at | 153.12 | 153.12 | 155.16 | 156.16 | 153.10 | 153.10 | 153.10 | 153.10 |
| 62 wt. % in | | | | | | | | |
| H₂0 | | | | | | | | |
| Na₄P₂O₇ | 43.58 | 54.47 | 66.24 | 77.77 | 43.57 | 43.57 | 43.57 | 43.57 |
| Water | 228.15 | 228.15 | 228.15 | 228.15 | 228.15 | 228.15 | 228.15 | 228.15 |
| Antiscalant | | | | | | | | |
| Antiscalant | | | | | | | | |
| weight¹ | | | | | | | | |
| Precipitation | 70 | 70 | 70 | 70 | 50 | 60 | 70 | 70 |
| Temp (° C.) | | | | | | | | |
| Dosing time² | 60 | 60 | 60 | 60 | 60 | 60 | 60 | 60 |
| End Temp³ | 25 | 25 | 25 | 25 | 25 | 25 | 25 | 25 |
| Rinse steps⁴ | 4 | 4 | 4 | 4 | 4 | 4 | 4 | 4 |
| Yield of | 61.97 | 47.21 | 28.37 | 1.01 | 60.57 | 60.87 | 62.23 | 61.38 |
| Sn₂P₂O₇, in | | | | | | | | |
| Grams | | | | | | | | |
| Yield of | 93.8% | 71.5% | 42.9% | 1.5% | 91.7% | 92.1% | 94.2% | 92.9% |
| Sn₂P₂O₇ in % | | | | | | | | |
| of theoretical | | | | | | | | |
| yield | | | | | | | | |
| Particle size⁵ | 158.74 | 128.47 | 193.28 | 34.03 | 123.52 | 165.27 | 244.72 | 157.11 |
| Boron PPM⁶ | 16 | 18 | | | 24 | 21 | 17 | 16 |

Part 2

| | Run 9 | Run 10 | Run 11 | Run 12 | Run 13 | Run 14 | Run 15 |
|---|---|---|---|---|---|---|---|
| Sn(BF₄)₂ at | 153.10 | 153.10 | 153.10 | 153.10 | 153.10 | 153.10 | 150.00 |
| 62 wt. % in H₂0 | | | | | | | |
| Na₄P₂O₇ | 43.57 | 43.57 | 43.57 | 43.57 | 43.57 | 43.57 | 42.69 |
| Antiscalant | | | | | | | Citric |
| | | | | | | | acid |
| Antiscalant | | | | | | | 0.228 |
| weight⁷ | | | | | | | |
| Precipitation | 70 | 70 | 70 | 70 | 70 | 70 | 70 |
| Temp | | | | | | | |
| Dosing time⁸ | 120 | 30 | 60 | 60 | 60 | 60 | 60 |
| End Temp⁹ | 25 | 25 | 40 | 10 | 22 | 22 | 22 |
| Rinse steps¹⁰ | 4 | 4 | 4 | 4 | 4 | 4 | 4 |
| Yield of | 62.11 | 62.45 | 61.77 | N/A | 61.00 | 60.63 | 64.2 |
| Sn₂P₂O₇, in | | | | | | | |
| Grams | | | | | | | |
| Yield of | 94.0% | 94.5% | 93.5% | N/A | 92.3% | 91.8% | 97.2% |
| Sn₂P₂O₇ in % of | | | | | | | |
| theoretical yield | | | | | | | |
| Particle size¹¹ | 193.17 | 183.29 | 142.86 | N/A | 105.01 | 114.57 | 8.38 |
| Boron PPM¹² | 14 | 17 | 13 | N/A | 10 | 5 | |

part 3

| | Run 15 | Run 16 | Run 17 | Run 18 | Run 19 | Run 20 | Run 21 |
|---|---|---|---|---|---|---|---|
| Sn(BF₄)₂ at | 150.00 | 150.00 | 150.00 | 150.00 | 150.00 | 150.00 | 150.00 |
| 62 wt. % in H₂0 | | | | | | | |
| Na₄P₂O₇ | 42.69 | 42.69 | 42.69 | 42.69 | 36.95 | 51.79 | 42.69 |
| Antiscalant¹³ | DTPMP | DTPMP | NTMP | HEDP | | | |
| Antiscalant | 0.114 | 0.228 | 0.228 | 0.228 | | | |
| weight in | | | | | | | |
| grams¹⁴ | | | | | | | |
| Precipitation | 70 | 70 | 70 | 70 | 70 | 70 | 70 |
| Temp | | | | | | | |
| Dosing time¹⁵ | 60 | 60 | 60 | 60 | 90 | 75 | 75 |
| End Temp¹⁶ | 22 | 22 | 22 | 22 | 22 | 22 | 22 |
| Rinse steps¹⁷ | 4 | 4 | 4 | 4 | 4 | 4 | 4 |
| Yield of | 64.2 | 62.1 | 63.2 | 62.8 | 56.65 | 61 | 61 |
| Sn₂P₂O₇, in | | | | | | | |
| Grams | | | | | | | |
| Yield of Sn₂P₂O₇ | 97.2% | 94.0% | 95.7% | 95.1% | 94.8% | 93.9% | 93.9% |
| in % of | | | | | | | |
| theoretical yield | | | | | | | |
| Particle size¹⁸ | 110.00 | 167.90 | 65.80 | 102.00 | 5.51 | 95.9 | 95.9 |
| Boron PPM¹⁹ | N/A | N/A | N/A | N/A | N/A | N/A | N/A |

¹Antiscalant weight in grams.
²Dosing time is the time for the addition of the tin tetrafluoroborate solution to the pyrophosphate salt solution.
³The “end temp” is the temperature at the time the precipitate was filtered out.
⁴All rinse steps used 70 milliliters of deionized water.
⁵Particle size of the dried precipitate.
⁶Concentration of the element Boron in the dried precipitate, in PPM by weight, based on the total weight of the dried precipitate.
⁷Antiscalant weight in grams.
⁸Dosing time is the time for the addition of the tin tetrafluoroborate solution to the pyrophosphate salt solution.
⁹The “end temp” is the temperature at the time the precipitate was filtered out.
¹⁰All rinse steps used 70 milliliters of deionized water.
¹¹Particle size of the dried precipitate.
¹²Concentration of the element Boron in the dried precipitate, in PPM by weight, based on the total weight of the dried precipitate.
¹³DTPMP is diethylenetriamine penta (methylene phosphonic acid), NTMP is nitrilotris(methylene) triphosphonic acid, and HEDP is etidronic acid.
¹⁴Antiscalant weight in grams
¹⁵Dosing time is the time for the addition of the tin tetrafluoroborate solution to the pyrophosphate salt solution.
¹⁶The “end temp” is the temperature at the time the precipitate was filtered out.
¹⁷All rinse steps used 70 milliliters of deionized water.
¹⁸Particle size of the dried precipitate.
¹⁹Concentration of the element Boron in the dried precipitate, in PPM by weight, based on the total weight of the dried precipitate.

As illustrated in the results presented above, higher yields are produced by approximately stoichiometric addition of the tin tetrafluoroborate 10 and the pyrophosphate salt compound 16. The use of antiscalants 24 can decrease particle size, where some antiscalants 24 are more effective than others. Increasing the addition period (referred to as the “dosing time” in Table 1) can reduce particle size, but results are not significant until the addition period is increased to several hours.

While several embodiments have been presented in the foregoing detailed description, it should be appreciated that a vast number of variations exist. It should also be appreciated that the embodiment or embodiments are only examples, and are not intended to limit the scope, applicability, or configuration of this disclosure in any way. Rather, the foregoing detailed description will provide those skilled in the art with a convenient road map for implementing various embodiments of the asphalt compositions, it being understood that various changes may be made in the function and arrangement of elements described without departing from the scope as set forth in the appended claims and their legal equivalents.

What is claimed is:

1. A method of producing tin pyrophosphate, the method comprising the steps of:
combining tin tetrafluoroborate and a pyrophosphate salt composition to produce a precipitate, wherein the precipitate comprises the tin pyrophosphate and a tetrafluoroborate salt; and
removing the tetrafluoroborate salt from the precipitate.

2. The method of claim 1, further comprising:
providing a tin tetrafluoroborate solution comprising the tin tetrafluoroborate and a tin tetrafluoroborate solvent, wherein the tin tetrafluoroborate solvent comprises water, and wherein the tin tetrafluoroborate solution is produced prior to combining the tin tetrafluoroborate and the pyrophosphate salt composition.

3. The method of claim 1, further comprising:
producing a pyrophosphate salt solution comprising the pyrophosphate salt composition and a pyrophosphate salt solvent, wherein the pyrophosphate salt solvent comprises water, and wherein combining the tin tetrafluoroborate and the pyrophosphate salt composition comprises combining the pyrophosphate salt solution and the tin tetrafluoroborate.

4. The method of claim 3, further comprising:
bringing the pyrophosphate salt solution to a pyrophosphate salt solution temperature of from about 60 to about 85 degrees Celsius (° C.) prior to combining the tin tetrafluoroborate and the pyrophosphate salt solution.

5. The method of claim 3, wherein:
combining the tin tetrafluoroborate and the pyrophosphate salt solution comprises adding the tin tetrafluoroborate to the pyrophosphate salt solution.

6. The method of claim 5, further comprising providing a tin tetrafluoroborate solution comprising the tin tetrafluoroborate and a tin tetrafluoroborate solvent, wherein combining the tin tetrafluoroborate with the pyrophosphate salt solution comprises adding the tin tetrafluoroborate solution to the pyrophosphate salt solution over an addition period of about 5 minutes to about 12 hours.

7. The method of claim 3, wherein the pyrophosphate salt solution further comprises phosphoric acid to provide a pyrophosphate salt solution pH of from about 9.5 to about 7.

8. The method of claim 3, wherein the pyrophosphate salt solution further comprises phosphoric acid in an amount sufficient to produce the precipitate with an average particle size of from about 0.1 to about 100 microns.

9. The method of claim 3, wherein the pyrophosphate salt solution further comprises an antiscalant in an amount of from about 0.01 to about 1 weight percent, based on a weight of the pyrophosphate salt solution, wherein the antiscalant is selected from a group of citric acid, nitrilotris(methylene) triphosphonic acid, etidronic acid, phosphonobutane tricarboxylic acid, ethylenediamine tetra (methylene phosphonic acid), hexamethylene diamine tetra (methylene phosphonic acid), diethylenetriamine penta (methylene phosphonic acid), and combinations thereof.

10. The method of claim 3, wherein the pyrophosphate salt solution further comprises an antiscalant in an amount sufficient to produce the precipitate with an average particle size of from about 0.1 to about 100 microns, wherein the antiscalant is selected from a group of citric acid, nitrilotris(methylene) triphosphonic acid, etidronic acid, phosphonobutane tricarboxylic acid, ethylenediamine tetra (methylene phosphonic acid), hexamethylene diamine tetra (methylene phosphonic acid), diethylenetriamine penta (methylene phosphonic acid), and combinations thereof.

11. The method of claim 1, wherein the pyrophosphate salt composition comprises tetrasodium pyrophosphate.

12. The method of claim 1, wherein:
the tin tetrafluoroborate is added in an amount of about 100 to about 125% of a stoichiometric amount for a reaction with the pyrophosphate salt composition.

13. The method of claim 1, wherein:
the tin tetrafluoroborate and the pyrophosphate salt composition are combined in a microjet reactor.

14. The method of claim 1 further comprising:
spray drying the precipitate.

15. The method of claim 1, wherein removing the tetrafluoroborate salt comprises rinsing the precipitate with a rinsate, wherein the rinsate comprises from about 50 to about 100 weight percent water, based on a total weight of the rinsate.

16. The method of claim 1, wherein removing the tetrafluoroborate salt comprises washing the precipitate until a boron concentration in the precipitate is from about 0.1 to about 100 parts per million by weight, based on a total weight of the precipitate.

17. The method of claim 1, further comprising:
drying the precipitate after removing the tetrafluoroborate salt.

18. A dental health care composition comprising:
tin pyrophosphate at a concentration of from about 96 to about 99.999 weight percent, based on a total weight of the dental health care composition; and
sodium tetrafluoroborate at a concentration of from about 10 to about 1,000 parts per million by weight, based on the total weight of the dental health care composition.

19. The dental health care composition of claim 18, wherein the tin pyrophosphate has an average particle size of from about 0.1 to about 100 microns.

20. A method of producing tin pyrophosphate, the method comprising the steps of:
providing a tin tetrafluoroborate solution comprising tin tetrafluoroborate and a tin tetrafluoroborate solvent, wherein the tin tetrafluoroborate solvent comprises water;
providing a pyrophosphate salt solution comprising tetrasodium pyrophosphate and a pyrophosphate salt solvent, wherein the pyrophosphate salt solvent comprises water;
adjusting a pyrophosphate salt solution temperature to about 60 to about 85 degrees Celsius (° C.),
combining the tin tetrafluoroborate solution and the pyrophosphate salt solution to produce a precipitate comprising the tin pyrophosphate and sodium tetrafluoroborate; and
rinsing the precipitate with a rinsate to reduce a sodium tetrafluoroborate concentration to a level such that a boron concentration is less than about 100 parts per million by weight, based on a total weight of the precipitate.
